(19) United States
(12) Patent Application Publication
(10) Pub. No.: US 2006/0247556 A1
(43) Pub. Date: Nov. 2, 2006

(54) Guide wire for use in a surgical procedure

(76) Inventor: Henry William Lupton, Oranmore (IE)

Correspondence Address: SUGHRUE MION, PLLC, 2100 PENNSYLVANIA AVENUE, N.W., SUITE 800, WASHINGTON, DC, 20037, US

(21) Appl. No.: 10/567,359

(22) PCT Filed: Aug. 6, 2004
(86) PCT No.: PCT/IE04/00106

§ 371 Date: Apr. 14, 2006

(30) Foreign Application Priority Data
Aug. 6, 2003 (IE) S2003/0579

(51) Int. Cl. A61M 25/00

(52) U.S. Cl. 600/585

(57) ABSTRACT

A guide wire (1) extending between a distal end (3) and a proximal end (4) defines a central axis (5). A main tubular member (6) of the guide wire (1) terminates in a curvature controllable portion (15) adjacent the distal end (3) for facilitating inducing a curvature therein for offsetting the distal end (3) at an angle (α₁,α₂) relative to the central axis (5). A first member (19) of the curvature controllable portion (15) and a second member (21) thereof are secured to a bulbous tip (7) at the distal end (3), and the second member (21) is slideable in the direction of the arrows A and C relative to the first member (19) for inducing curvature in the curvature controllable portion (15) for in turn offsetting the distal end (3) in the direction of the arrows B and D, respectively, from the central axis (5). A connecting wire (35) terminating in an operating member (17) is connected to and extends from the second member (21) for facilitating urging of the second member (21) in the direction of the arrows A and C relative to the first member (19).

The present invention relates to a guide wire for use in a surgical or other procedure for accessing a remote site in the body of a human or animal subject, and in particular though not limited to a guide wire for use with a catheter. The invention also relates to a method for forming a guide wire, and the invention relates to a catheter and the guide wire in combination, and to a guide wire for use in accessing a remote site in the body of a human or animal subject.

Guide wires for locating a distal end of a catheter in a remote site in the body of a human or animal subject are known. Such guide wires are commonly used for guiding a catheter along narrow blood vessels to a site in the cardiovascular system of the subject for enabling cardiovascular procedures to be carried out. Typically, the guide wire is introduced through a cannula into a suitable blood vessel in the thigh or arm of the subject and is passed through the blood vessels to the desired site in the cardiovascular system. Once the guide wire has reached the desired site, the catheter is then advanced over the guide wire to the site. Guide wires are also extensively used to guide a catheter to other sites through the vascular system, and also to sites in the renal system, as well as to other sites in human and animal subjects through other accessing systems. Indeed, guide wires are also used for accessing a remote site in the body of a human or animal subject for purposes other than for guiding a catheter to the remote site.

Due to the relatively narrow diameter of the blood vessels and other vessels through which the guide wire has to pass, and in particular, due to the tortuous nature of the blood vessels of the vascular and other systems, and the number of branching blood vessels in the vascular system, it is essential that the guide wire be of a construction which facilitates easy alignment of the distal end of the guide wire with a desired one of branching blood vessels, through which the guide wire is to be passed, as the guide wire is being advanced through the vascular or other system, so that further urging of the guide wire into the vascular or other system results in the guide wire entering the desired branched vessel, and passing therethrough.

Various attempts have been made to provide a guide wire the distal end of which can be aligned with and urged into a branching blood vessel. For example, it is known to provide a guide wire with a distal portion which is readily bendable into a desired curved configuration for offsetting the distal end of the guide wire at an angle relative to a central axis of the guide wire so that by rotating the guide wire within the vascular or other system, the distal end thereof can be aligned with a branching blood vessel through which it is desired to pass the guide wire, so that further advancing of the guide wire through the vascular system urges the guide wire into and through the branching blood vessel. However, such guide wires require that the bend be formed in the distal portion of the guide wire prior to entering the guide wire into the vascular or other system of the subject, and furthermore, once the distal portion of the guide wire is bent, it cannot be subsequently straightened as the guide wire is being advanced through the vascular or other system of the subject. This is particularly undesirable where a guide wire is to be used in the vascular system where the blood vessels of varying cross-section, and in particular, where the blood vessels are of relatively small cross-section, since the effective cross-section of the guide wire is increased by virtue of the bend imposed in the bendable distal portion. This is a particular problem in the cardiovascular system, where the blood vessels are of relatively small cross-section.

Efforts to overcome this problem have resulted in guide wires which facilitate remote selective bending of the guide wire, so that the distal portion may be bent and straightened as the guide wire is being advanced through the vascular or other systems of the subject. One such guide wire is disclosed in U.S. Pat. No. 3,802,440 of Salem, et al. The guide wire of Salem comprises a tubular guide wire which extends between a proximal and a distal end. A plurality of spaced apart transversely extending slots are formed to one side of the tubular guide wire at a distal portion thereof for facilitating bending of the guide wire at the distal portion, for in turn offsetting the distal end of the guide wire at an angle relative to a central axis defined by the guide wire. A rod which is secured to the distal end of the guide wire extends axially within the bore of the tubular guide wire to the proximal end of the guide wire, and is connected to an operating lever located at the proximal end of the guide wire. By urging the rod axially within the tubular guide wire and relative to the tubular guide wire, curvature is induced in the distal portion, with the distal tip being urged away from the central axis in a direction towards and away from the side of the guide wire in which the transverse slots are formed.

However, a problem with the guide wire of Salem is that it is difficult to control the curvature of the bend induced in the distal portion by relative movement of the rod and the tubular guide wire. Due to the non-uniformity of the strength of the tubular member in the distal portion resulting from the spaced apart transverse slots, bending of the distal portion may occur around one of the transverse slots only, or around a few, or all of the transverse slots. Thus, depending on whether the bending of the distal portion occurs around one or more of the transverse slots, the induced curvature will be more or less acute. If the curvature occurs around one of the transverse slots only, the curvature will be quite acute, while if the curvature is induced along a number of the slots, the curvature will be less acute. Even where the curvature occurs along all the transverse slots, it is still difficult to maintain uniform curvature, since the distal portion may bend at a more acute curvature around one or more of the transverse slots, as it does around others of the transverse slots. This is undesirable, since it permits limited control of the angle by which the distal tip is offset relative to the central axis of the guide wire. In general, it is desirable that the angle at which the distal end is offset relative to the central axis of the guide wire is relatively accurately controllable for facilitating easy alignment of the distal end with a branching blood vessel.

There is therefore a need for a guide wire which permits relatively accurate control of the curvature induced in a curvature controllable portion of a guide wire, for in turn relatively accurately controlling the angle at which the distal end of a guide wire is offset from a central axis of the guide wire, as the guide wire is being advanced through the body of a human or animal subject to a remote site therein.

The present invention is directed towards providing such a guide wire, and the invention is also directed towards providing a method for forming such a guide wire, as well as a catheter and the guide wire in combination, and the invention is further directed towards providing a guide wire for use in accessing a remote site in the body of a human or animal subject.

According to the invention there is provided an elongated guide wire for use in a surgical or other procedure for accessing a remote site in the body of a human or animal subject, the guide wire defining a longitudinally extending central axis, and extending between a distal end for accessing the remote site and a spaced apart proximal end, a curvature controllable portion being located in the guide wire towards the distal end thereof for offsetting the distal end at an angle relative to the central axis, the curvature controllable portion comprising an elongated curvature inducing first member, and an elongated curvature inducing second member coupled to each other adjacent their distal ends, and extending from their distal ends axially in a proximal direction, and being moveable axially relative to each other for inducing a curved bend in the curvature controllable portion, wherein a means is provided for constraining the first and second members to move parallel to each other for inducing the curved bend in the curvature controllable portion.

In one embodiment of the invention the first and second members are disposed side by side and are slideably moveable axially relative to each other.

In one embodiment of the invention the means for constraining the first and second members to move parallel to each other comprises a guide tongue extending laterally from the second member and being slideably engageable with an axially extending corresponding guide groove in the first member.

Preferably, a retaining means is provided for retaining the guide tongue laterally captive in the guide groove.

Advantageously, the retaining means is provided for retaining the guide tongue laterally captive in a plane in which curvature is induceable in the curvature controllable portion.

In one embodiment of the invention the retaining means is provided by the transverse cross-section of the guide tongue co-operating with the transverse cross-section of the guide groove.

In another embodiment of the invention the guide tongue extends from the second member in a plane parallel to the plane in which curvature is induceable in the curvature controllable portion.

Preferably, the guide groove is of transverse cross-section which defines the transverse cross-section of the guide tongue. Advantageously, the guide groove is formed by an elongated guide bore extending axially in the first member, and by a guide slot extending axially along the first member for accommodating the guide tongue into the guide bore. Ideally, the transverse width of the guide slot is less than a transverse dimension of the guide bore parallel to the transverse width of the guide slot for retaining the guide tongue laterally captive in the guide groove.

In one embodiment of the invention the guide bore is of circular transverse cross-section.

In another embodiment of the invention the guide tongue comprises a guide member slideable in the guide bore, and a coupling member extending through the guide slot for coupling the guide member to the second member.

Preferably, the guide member extends axially the length of the second member, and preferably, the coupling member extends axially the length of the second member.

In one embodiment of the invention one of the first and second members is of axial length shorter than the axial length of the other of the first and second members for accommodating relative axial movement of one of the first and second members relative to the other, and preferably, the axial length of the second member is shorter than the axial length of the first member.

Preferably, the first and second members define a transverse cross-sectional area having a circular outer periphery.

In one embodiment of the invention the guide wire comprises an elongated tubular member extending from the proximal end to the distal end.

In another embodiment of the invention the first member is formed by the tubular member of the guide wire.

Preferably, an operating means is provided at the proximal end of the guide wire for moving one of the first and second members relative to the other for inducing the curved bend in the curvature controllable portion, and advantageously, a connecting means is provided for connecting the operating means to one of the first and second members.

In one embodiment of the invention the connecting means is connected to the second member. Preferably, the connecting means extends through a bore defined by the tubular member forming the guide wire, and advantageously, the connecting means co-operates with the tubular member forming the guide wire, so that the column strength of the connecting means is sufficient for facilitating urging of the second member relative to the first member in both axial directions.

In one embodiment of the invention the connecting means comprises an elongated connecting wire, and the operating means is formed by a portion of the connecting wire extending from the tubular member forming the guide wire, at the proximal end thereof for facilitating urging the guide wire in at least one axial direction for urging the second member in the corresponding axial direction relative to the first member.

In another embodiment of the invention the tubular member forming the guide wire is of a polymer material.

In a further embodiment of the invention the curvature controllable portion is located adjacent the distal end of the guide wire, and preferably, the distal end of the first and second members coincides with the distal end of the guide wire.

In another embodiment of the invention at least one of the first and second members is of resilient material for resiliently urging the distal end of the guide wire into axial alignment with the central axis of the guide wire.

In a further embodiment of the invention the first member is of a polymer material, and in another embodiment of the invention the second member is of a polymer material.

In one embodiment of the invention the distal end of the guide wire terminates in a bulbous tip.

In another embodiment of the invention the bulbous tip is formed by material used for securing the first and second members of the curvature controllable portion together. Preferably, the bulbous tip defines a hemispherical distal tip.

In one embodiment of the invention a sleeve extends from the distal end of the guide wire axially in a direction towards the proximal end thereof, and the curvature controllable portion being located within the sleeve, and preferably, the sleeve extends from the distal end to a location intermediate the distal end and the proximal end of the guide wire.

In one embodiment of the invention the sleeve is formed by a helically wound coil, and preferably, the sleeve is formed by a tightly wound helical coil.

In one embodiment of the invention the sleeve is of a metallic material, and in an alternative embodiment of the invention the sleeve is of a plastics material. In a further embodiment of the invention the sleeve is of a plastics material and a metallic material.

Preferably, at least a portion of the sleeve is of a radiopaque material adjacent the distal end of the guide wire.

In one embodiment of the invention at least a portion of the guide wire adjacent the distal end thereof is of a radiopaque material.

In one embodiment of the invention the degree of curvature induced in the curvature controllable portion is proportional to the amount of relative movement between the first and second members, and preferably, the angle by which the distal end of the guide wire is offset relative to the central axis of the guide wire is proportional to the amount of relative movement between the first and second members.

The invention also provides a guide wire according to the invention for use in accessing a remote site in the body of a human or animal subject.

The invention also provides in combination a catheter for use in a surgical or other procedure for accessing a remote site in the body of a human or animal subject, and an elongated guide wire, wherein the guide wire is a guide wire according to the invention.

Further the invention provides in combination a catheter for use in a surgical or other procedure for accessing a remote site in the body of a human or animal subject, and an elongated guide wire, the guide wire defining a longitudinally extending central axis, and extending between a distal end for accessing the remote site and a spaced apart proximal end, a curvature controllable portion being located in the guide wire towards the distal end thereof for offsetting the distal end at an angle relative to the central axis, the curvature controllable portion comprising an elongated curvature inducing first member, and an elongated curvature inducing second member coupled to each other adjacent their distal ends, and extending from their distal ends axially in a proximal direction, and being moveable axially relative to each other for inducing a curved bend in the curvature controllable portion, wherein a means is provided for constraining the first and second members to move parallel to each other for inducing the curved bend in the curvature controllable portion.

The invention also provides a method for forming an elongated guide wire for use in a surgical or other procedure for accessing a remote site in the body of a human or animal subject, the method comprising the steps of providing a guide wire defining a longitudinally extending central axis and extending between a distal end for accessing the remote site and a spaced apart proximal end, locating a curvature controllable portion in the guide wire towards the distal end thereof for offsetting the distal end at an angle relative to the central axis, and providing the curvature controllable portion with an elongated curvature inducing first member and an elongated curvature inducing second member coupled to each other adjacent their distal ends, so that the first and second members extend from their distal ends axially in a proximal direction, and are moveable axially relative to each other for inducing a curved bend in the curvature controllable portion, wherein a means is provided for constraining the first and second members to move parallel to each other for inducing the curved bend in the curvature controllable portion.

Preferably, the first and second members are disposed side by side and are slideably moveable axially relative to each other. Advantageously, the means for constraining the first and second members to move parallel to each other is provided by a guide tongue extending laterally from the second member and being slideably engageable with an axially extending corresponding guide groove in the first member.

The advantages of the invention are many. The angle at which the distal end of the guide wire is offset relative to the central axis of the guide wire can be relatively accurately controlled as the guide wire is being advanced through the body of a subject to a remote site, and thus, the distal end of the guide wire can be readily easily aligned with a branched vessel into which the guide wire is to be passed, as it is being advanced to the remote site. By virtue of the fact that the curvature inducing first and second members are constrained to move parallel to each other, the degree of curvature induced in the curvature controllable portion is proportional to the amount by which the first and second members are moved axially relative to each other, and thus, the curvature induced in the curvature controllable portion can be relatively accurately controlled. Additionally, the angle at which the distal end of the guide wire is offset relative to the central axis of the guide wire is proportional to the degree of curvature induced in the curvature controllable portion, and accordingly, the angle at which the distal end of the guide wire is offset relative to the central axis thereof is also proportional to the amount by which the first and second members are moved axially relatively to each other. Since the amount by which the first and second members are moved axially relative to each other can be accurately controlled by the connecting means, which connects the second member to the operating means, the angle at which the distal end of the guide wire is offset relative to the central axis of the guide wire can readily easily be controlled by controlling the amount by which the connecting means is urged relative to the tubular member forming the guide wire. Thus, in the guide wire according to the invention there is a direct correlation between the angle by which the distal end of the guide wire is offset relative to the central axis thereof and the amount by which the connecting means is urged relative to the tubular member forming the guide wire. Accordingly, any desired angle of offset of the distal end of the guide wire relative to the central axis thereof can be selected by appropriately urging the connecting means by the operating means into or out of the tubular member of the guide wire.

Thus, ready, easy and relatively accurate setting of the angle of offset of the distal end of the guide wire relative to the central axis thereof is permitted, which can be carried out remotely while the guide wire is being advanced through the vascular or other system of a subject. Furthermore, once the distal end of the guide wire has been advanced into a branching blood vessel, the curvature controllable portion can be readily straightened, thereby returning the distal end of the guide wire to coincide with the central axis of the guide wire. In embodiments of the invention whereby the connecting means co-operates with the tubular member forming the guide wire to be of sufficient column strength for facilitating urging of the second member relative to the first member in both directions, the distal end of the guide wire can be returned to coincide with the central axis of the guide wire by urging the second member relative to the first member in the opposite direction by the connecting means. Alternatively, where one or both of the first and second members are of resilient material, and are resiliently urged for returning the distal end of the guide wire to coincide with the central axis thereof, on releasing the connecting member, the resilient one or both of the first and second members resiliently returns the distal end of the guide wire to coincide with the central axis of the guide wire.

By providing the second member with a laterally extending guide tongue for engaging an axially extending corresponding guide groove in the first member, the first and second members are constrained to move parallel to each other, and furthermore, by providing the guide tongue and guide groove to co-operate so that the guide tongue is retained laterally captive in the guide groove, parallel relative movement of the first and second members is ensured. Additionally, by providing the second member with the guide tongue and the first member with the guide groove, a relatively convenient and relatively easily manufactured guide wire is provided.

The invention will be more clearly understood from the following description of a preferred embodiment thereof, which is given by way of example only, with reference to the accompanying drawings, in which:

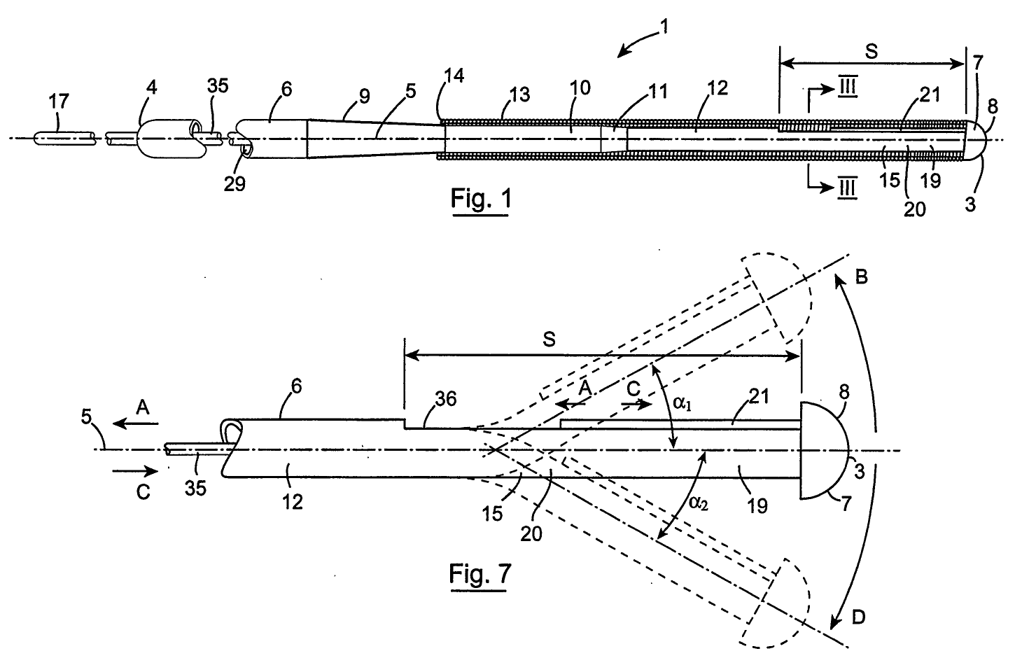

FIG. 1 is a side elevational view of a guide wire according to the invention,

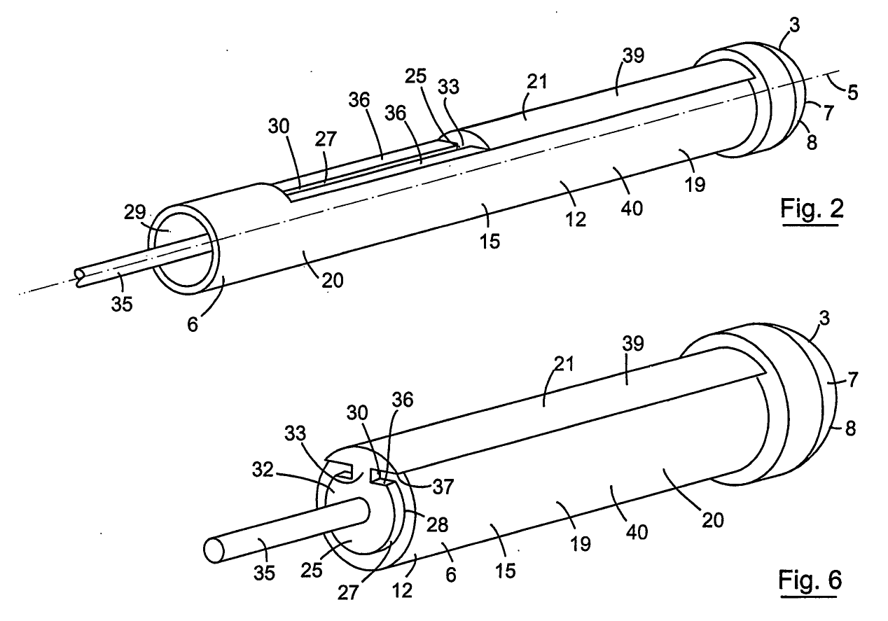

FIG. 2 is a perspective view of a portion of the guide wire of FIG. 1,

FIG. 3 is a transverse cross-sectional end elevational view of the guide wire of FIG. 1 on the line III-III of FIG. 1,

FIG. 4 is a perspective view of a portion of the guide wire of FIG. 1,

FIG. 5 is a perspective view of the portion of FIG. 4 illustrated in a different position,

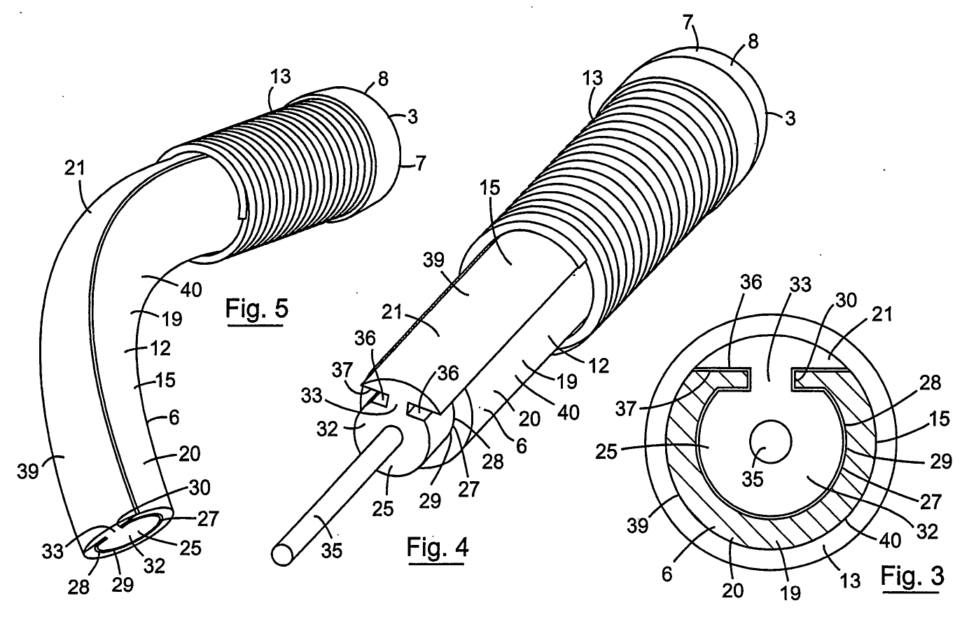

FIG. 6 is a perspective view of a detail of the guide wire of FIG. 1, and

FIG. 7 is a side elevational view of the portion of the guide wire of FIG. 2 illustrated in use.

Referring to the drawings, which are not to scale, there is illustrated a guide wire according to the invention, indicated generally by the reference numeral 1, for use with a catheter (not shown) for guiding the catheter to a remote site in the body of a human or animal subject. The guide wire 1 is particularly suitable for accessing a remote site in the cardiovascular system of the subject for, in turn, guiding a catheter to a remote site in the cardiovascular system, for example, for guiding a catheter to a remote site in the heart of a subject. However, it will be readily apparent to those skilled in the art that the guide wire 1 is suitable also for accessing any remote site in a human or animal subject, be it in the vascular system, the renal system, or other system. For example, the guide wire 1 is also suitable for accessing renal vessels, neuro-vasculature systems, the fallopian tubes, and other such vessels and sites.

The guide wire 1 extends between a distal end 3 and a proximal end 4, and defines a longitudinally extending central axis 5. The guide wire 1 comprises an elongated main tubular member 6 of flexible resilient material, in this embodiment of the invention a polymer material which extends from the proximal end 4 to the distal end 3. The main tubular member 6 terminates at the distal end 3 in a bulbous tip 7 to which it is secured. The bulbous tip 7 defines a hemispherical surface 8, which in turn forms a leading distal surface of the guide wire 1 for advancing through the vascular or other system of the subject for minimising damage to blood vessels or other vessels through which the guide wire 1 is being urged. In this embodiment of the invention the main tubular member 6 tapers at 9 to a parallel portion 10, and 11 to a parallel portion 12 which extends to the distal end 3.

A flexible sleeve 13, which in this embodiment of the invention is provided by a tightly wound helical coil extends axially from the bulbous tip 7 in a proximal direction, and terminates at a location 14 along the main tubular member 6 intermediate the distal end 3 and the proximal end 4, but closer to the distal end 3. The helical coil sleeve 13 is secured to the main bulbous tip 7 and to the main tubular member 6 at the location 14. In this embodiment of the invention the helical coil sleeve 13 is formed from a radiopaque material, namely, platinum alloy material, for facilitating imaging of the guide wire 1 as it is being advanced through the vascular or other system of a subject.

A curvature controllable portion 15 is formed in the main tubular member 6 adjacent the distal end 3, and is located within the helical coil sleeve 13, for facilitating remotely inducing curvature therein for in turn offsetting the distal end 3 of the guide wire 1 at an angle α₁ or α₂ relative to the central axis 5, see FIG. 7. An operating means, in this embodiment of the invention provided by an operating member 17 located at the proximal end 4 of the guide wire 1 is provided for selectively inducing a desired degree of curvature in the curvature controllable portion 15 as will be described below.

The curvature controllable portion 15 extends for a distance S in a proximal direction from the distal end 3, and comprises an elongated curvature inducing first member 19 which is formed by a distal portion 20 of the parallel portion 12 of the main tubular member 6, and which extends for the distance S from the distal end 3. An elongated curvature inducing second member 21 also of polymer material is coupled to the distal end 3 of the main tubular member 6 by being secured to the bulbous tip 7, and the second member 21 is located side by side with the first member 19 and extends parallel to the first member 19. Additionally, the second member 21 is slideably moveable axially relative to and parallel to the first member 19 for inducing curvature in the curvature controllable portion 15. As the second member 21 is urged in the direction of the arrow A relative to the first member 19, curvature is induced in the curvature controllable portion 15 for offsetting the distal end 3 of the guide wire 1 at an angle α₁ relative to the central axis 5 in the direction of the arrow B from the central axis 5, see FIG. 7. Urging of the second member 21 in the direction of the arrow C relative to the first member 19 induces curvature in the curvature controllable portion 15 for offsetting the distal end 3 of the guide wire 1 at an angle α₂ relative to the central axis 5 in the direction of the arrow D from the central axis 5, see FIG. 7.

A means for constraining the first and second members 19 and 21 to move axially parallel relative to each other comprises an elongated guide tongue 25 extending laterally from the second member 21 for slideably engaging an elongated guide groove 27 extending axially in the first member 19. The guide groove 27 is formed by a guide bore 28, which in turn is formed by a main bore 29 defined by the main tubular member 6. An elongated guide slot 30 extends axially along the first member 19 and communicates with the guide bore 28 for accommodating the guide tongue into the guide bore 28. The guide tongue 25 comprises an elongated guide member 32 which is axially slideable in the guide bore 28, and is coupled to the second member 21 by a coupling member 33 extending between the second member 21 and the guide member 32, and which is slideable in the guide slot 30. In this embodiment of the invention the guide member 32 is of substantially circular transverse cross-section, and the transverse cross-section of the guide bore 28 and the guide slot 30 define the transverse cross-section of the guide member 32 and the coupling member 33, so that the guide member 32 and the coupling member 33 form a smooth sliding fit in the guide bore 28 and the guide slot 30 for facilitating relative parallel sliding movement between the first and second members 19 and 21. In this embodiment of the invention the guide member 32 and the coupling member 33 are of polymer material, and are integrally formed with the second member 21. The guide member 32 of the guide tongue 25 co-operates with the guide bore 28 of the guide groove 27 to act as a retaining means for retaining the guide tongue 25 laterally captive in the guide groove 27, and particularly laterally captive in a plane in which the curvature is formed in the curvature controllable portion 15 and in which the distal tip 3 is offset relative to the central axis 5 in the directions of the arrows B and D, see FIG. 7, for in turn constraining the second member 21 to move axially parallel relative to the first member 19.

The operating member 17 is coupled to the second member 21 for urging the second member 21 axially relative to the first member 19 for inducing, curvature in the curvature controllable portion 15 by a connecting means, namely, an elongated connecting wire 35. The connecting wire 35 is also of a polymer material, and is secured to the guide member 32 by adhesive. The connecting wire 35 extends axially through the main bore 29 of the main tubular member 6, and exits the main bore 27 at the proximal end 4 to form the operating member 17. In this embodiment of the invention the diameter of the connecting wire 35 is such, and the connecting wire 35 co-operates with the main tubular member 6 so that the column strength of the connecting wire 35 is such as to facilitate urging of the second member 21 in the directions of the arrows A and C relative to the first member 19, for facilitating offsetting the distal Up 3 of the guide wire 1 in the directions of the respective arrows B and D, on respective opposite sides of the central axis 5, and also for returning the distal end 3 from an offset position to coincide with the central axis 5 of the guide wire 1.

By virtue of the fact that the second member 21 is constrained by the co-operating action of the guide tongue 25 and the guide groove 27 to move parallel relative to the first member 19, the degree of curvature induced in the curvature controllable portion 15 is proportional to the amount by which the operating member 17 of the connecting wire 25 is urged into or out of the main bore 29 of the main tubular member 6. Since the angle by which the distal end 3 of the guide wire 1 is offset relative to the central axis 5 is proportional to the curvature induced in the curvature controllable portion 15, the angle by which the distal end 3 is offset relative to the central axis 5 is similarly proportional to the amount by which the operating member 17 of the connecting wire 35 is urged Into or out of the main bore 29 of the main tubular member 6. Therefore, the value of the angle α₁ and α₂ by which the distal end is offset from the central axis 5 is readily selectable by selecting the appropriate amount by which the operating member 17 is urged into or out of the main tubular member 6.

In this embodiment of the invention the first member 19 is formed from the main tubular member 6 by removing a portion of the material from the main tubular member 6 to form a flat bearing surface 36 for slideably engaging a corresponding flat bearing surface 37 of the second member 21. The guide slot 30 is machined into the guide bore 28 through the flat bearing surface 36. The second member 21 is of transverse cross-section, so that the combined transverse cross-sectional area of the first member 19 and the second member 21 define a circular periphery 39 which coincides with the periphery 40 of the main tubular member 6. The guide member 32 and the coupling member 33 which form the guide tongue 35 extend the length of the second member 21. However, the second member 21 is of shorter axial length than the axial length of the first member 19 in order to facilitate relative movement of the second member 21 relative to the first member 19 for inducing the curvature in the curvature controllable member 15.

In this embodiment of the invention the bulbous tip 7 is machined from polymer material, similar to the polymer material of the main tubular member 6 and is secured to the main tubular member 6 by adhesive. The bulbous tip 7 is secured to the second member 21 by adhesive. The helical coil sleeve 13 is secured to the bulbous tip 7 and the main tubular member 6 at the location 14 by adhesive.

In use, with the curvature controllable portion 15 extending straight and with the distal end 3 coinciding with the central axis 5, the guide wire 1 is inserted into an appropriate vein or artery of the subject and advanced through the vessels thereof. When it is desired to enter a branched blood vessel, the distal end 3 of the guide wire 1 is offset by a desired angle α₁ or α₂ relative to the central axis 5 by inducing a corresponding curve into the curvature controllable portion 15. This is achieved by urging the operating member 17 into or out of the main bore 29 of the main tubular member 6 by an appropriate amount to obtain the desired degree of curvature in the curvature controllable portion 15. When it is desired to offset the distal end 3 in the direction of the arrow B from the central axis 5, see FIG. 7, the operating member 15 is urged out of the main bore 29 of the main tubular member 6 an appropriate amount in the direction of the arrow A, for in turn urging the second member 21 in the direction of the arrow A relative to the first member 19. Alternatively, when it is desired to offset the distal end 3 in the direction of the arrow D from the central axis 5, the operating member 17 is urged into the main bore 29 of the main tubular member 6 in the direction of the arrow C, for in turn urging the second member 21 relative to the first member 19 in the direction of the arrow C.

The operating member 17 is urged into or out of the main bore 29, as the case may be, by an appropriate amount in order to obtain the desired degree of curvature in the curvature controllable portion 15, and in turn the desired value of the offset angle α₁ or α₂. If when the distal end 3 of the guide wire 1 has been offset by the desired angle α₁ or α₂, relative to the central axis 5, the distal end 3 is not aligned with the branching blood vessel, the guide wire 1 is rotated within the vascular system until the distal end 3 is aligned with the branching blood vessel. At that stage the guide wire is then urged further into the vascular or other system of the subject, thereby entering the distal end 3 into the branched blood vessel.

Once the distal end 3 has been urged into the branched blood vessel, the curvature controllable portion 15 can be straightened for returning the distal end 3 to coincide with the central axis 5. This is achieved by urging the operating member 17 in the opposite direction either into or out of, as the case may be, the main bore 29 of the main tubular member 6, in order to straighten the curvature controllable portion 15. Further urging of the guide wire 1 into the vascular or other system of the subject advances the guide wire 1 through the branched blood vessel.

Additionally, should a bend in a blood vessel be encountered, in order to ease the guide wire 1 around the bend, curvature can again be induced into the curvature controllable portion 15 as already described, and the distal end 3 of the guide wire 1 urged around the bend.

Ideally, the operating member 17 extending from the connecting wire 35 would be graduated to indicate the amount of angular offset of the distal end 3 of the guide wire 1 relative to the central axis 5 corresponding to units of movement of the operating member 17.

While the operating member 17 has been described as being provided by a portion of the connecting wire 35 extending from the main tubular member 6, at the proximal end thereof, it is envisaged in certain cases that an operating mechanism may be provided at the proximal end of the guide wire which would include a pivotally mounted lever coupled to the operating member 7, or directly to the connecting wire 35, so that pivotal movement of the lever would cause the connecting wire 35 to be urged in the direction of the arrows A and C, relative to the main tubular member 6. Where such an operating mechanism is provided, it is envisaged that the lever may be moveable over a graduated angular scale, which would indicate the amount of angular movement of the distal end 3 of the guide wire 1 relative to the central axis 5 corresponding to units of angular movement of the lever.

While the guide wire has been described as being provided with a sleeve formed by a tightly wound helical coil of a particular material, the helical coil may be of any other suitable or desirable material, and while it is preferable that it be of a radiopaque material, this is not essential. Additionally, the sleeve instead of being provided by a helical coil may be provided by a tubular member, for example, a tubular member of plastics or polymer material. Indeed, in certain cases it is envisaged that the sleeve may be provided by a combination of a helical coil and a tubular sleeve, whereby the sleeve would be alternately provided along its axial length by a helical coil and a tubular sleeve. Indeed, in certain cases the sleeve may be dispensed with.

It will also be appreciated that the main tubular member of the guide wire may be of any other material besides a polymer material, and indeed, in many cases, the main tubular member may be of a metallic material, for example, stainless steel, platinum alloy, nickel titanium alloy or the like. It is also envisaged that the second member may be of any other material besides a polymer material, and may be of a metallic material, which may be similar or different to that of the main tubular member forming the guide wire.

While the first member has been described as being integrally formed with and from the main tubular member, it is envisaged that the first member may be formed separately and secured to the main tubular member, for example, by bonding, brazing, welding, soldering or the like, depending on the material of the first member and the main tubular member.

While the connecting wire has been described as being of a particular material, the connecting wire may be of any other suitable material, for example, stainless steel, platinum alloy, nickel titanium alloy, or any other suitable material, and while the connecting wire has been described as being of material and co-operating with the main tubular member 6 to provide sufficient column strength to the connecting wire 35 in order to urge the second member in both directions relative to the first member, while this is desirable, it is not essential. Where either the first or second, or both the first and second members are provided of resilient material, and which in their normal state extend straight, after a curve being induced in the curvature controllable portion by pulling the connecting wire, on release of the connecting wire the first and second members would be resiliently urged back to their straight position with the distal end of the guide wire aligned with the central axis thereof. Indeed, in certain cases a return means for resiliently returning the curvature controllable member to its straight state, for in turn resiliently returning the distal tip to coincide with the central axis of the guide wire may be provided, such a return means may be provided by a spring or other such resilient means acting between the second member and the first member, or acting between the second member and the main tubular member forming the guide wire.

While a particular shape and construction of guide tongue and guide groove have been described for retaining the first and second members to move relative to each other with parallel axial sliding movement, guide tongues and guide grooves of other shape and construction could be provided.

Furthermore, while a guide tongue and a guide groove have been described for constraining the first and second members to move parallel to each other, any other suitable means for constraining the first and second members to move parallel to each other may be provided.

While the guide wire has been described for use in guiding a catheter to a remote site, the guide wire may be used for any other purpose, and indeed, in certain cases, it is envisaged that the guide wire may be used without a catheter.

1.-51. (canceled)

52. An elongated guide wire for use in a surgical or other procedure for accessing a remote site in the body of a human or animal subject, the guide wire defining a longitudinally extending central axis, and extending between a distal end for accessing the remote site and a spaced apart proximal end, a curvature controllable portion being located in the guide wire towards the distal end thereof for offsetting the distal end at an angle relative to the central axis, the curvature controllable portion comprising an elongated curvature inducing first member, and an elongated curvature inducing second member coupled to each other adjacent their distal ends, and extending from their distal ends axially in a proximal direction, and being moveable axially relative to each other for inducing a curved bend in the curvature controllable portion, wherein a means is provided for constraining the first and second members to move parallel to each other for inducing the curved bend in the curvature controllable portion.

53. A guide wire as claimed in claim 52 in which the first and second members are disposed side by side and are slideably moveable axially relative to each other.

54. A guide wire as claimed in claim 52 in which the means for constraining the first and second members to move parallel to each other comprises a guide tongue extending laterally from the second member and being slideably engageable with an axially extending corresponding guide groove in the first member.

55. A guide wire as claimed in claim 54 in which a retaining means is provided for retaining the guide tongue laterally captive in a plane in which curvature is induceable in the curvature controllable portion.

56. A guide wire as claimed in claim 55 in which the retaining means is provided by the transverse cross-section of the guide tongue co-operating with the transverse cross-section of the guide groove.

57. A guide wire as claimed in claim 52 in which the guide wire comprises an elongated tubular member extending from the proximal end to the distal end, and the first member is formed by the tubular member of the guide wire.

58. A guide wire as claimed in claim 57 in which an operating means is provided at the proximal end of the guide wire for moving one of the first and second members relative to the other for inducing the curved bend in the curvature controllable portion, and a connecting means is provided for connecting the operating means the second member.

59. A guide wire as claimed in claim 58 in which the connecting means extends through a bore defined by the tubular member forming the guide wire.

60. A guide wire as claimed in claim 58 in which the connecting means co-operates with the tubular member forming the guide wire, so that the column strength of the connecting means is sufficient for facilitating urging of the second member relative to the first member in both axial directions.

61. A guide wire as claimed in claim 58 in which the connecting means comprises an elongated connecting wire, and the operating means is formed by a portion of the connecting wire extending from the tubular member forming the guide wire, at the proximal end thereof for facilitating urging the guide wire in at least one axial direction for urging the second member in the corresponding axial direction relative to the first member.

62. A guide wire as claimed in claim 52 in which at least one of the first and second members is of resilient material for resiliently urging the distal end of the guide wire into axial alignment with the central axis of the guide wire.

63. A guide wire as claimed in claim 52 in which the distal end of the guide wire terminates in a bulbous tip.

64. A guide wire as claimed in claim 52 in which a sleeve extends from the distal end of the guide wire axially in a direction towards the proximal end thereof, and the curvature controllable portion is located within the sleeve.

65. A guide wire as claimed in claim 52 in which at least a portion of the guide wire adjacent the distal end thereof is of a radiopaque material.

66. A guide wire as claimed in claim 52 for use in accessing a remote site in the body of a human or animal subject.

67. In combination a catheter for use in a surgical or other procedure for accessing a remote site in the body of a human or animal subject, and an elongated guide wire, characterised in that the guide wire is a guide wire as claimed in claim 52.

68. In combination a catheter for use in a surgical or other procedure for accessing a remote site in the body of a human or animal subject, and an elongated guide wire, the guide wire defining a longitudinally extending central axis, and extending between a distal end for accessing the remote site and a spaced apart proximal end, a curvature controllable portion being located in the guide wire towards the distal end thereof for offsetting the distal end at an angle relative to the central axis, the curvature controllable portion comprising an elongated curvature inducing first member, and an elongated curvature inducing second member coupled to each other adjacent their distal ends, and extending from their distal ends axially in a proximal direction, and being moveable axially relative to each other for inducing a curved bend in the curvature controllable portion, characterised in that a means is provided for constraining the first and second members to move parallel to each other for inducing the curved bend in the curvature controllable portion.

69. A method for forming an elongated guide wire for use in a surgical or other procedure for accessing a remote site in the body of a human or animal subject, the method comprising the steps of providing a guide wire defining a longitudinally extending central axis and extending between a distal end for accessing the remote site and a spaced apart proximal end, locating a curvature controllable portion in the guide wire towards the distal end thereof for offsetting the distal end at an angle relative to the central axis, and providing the curvature controllable portion with an elongated curvature inducing first member and an elongated curvature inducing second member coupled to each other adjacent their distal ends, so that the first and second members extend from their distal ends axially in a proximal direction, and are moveable axially relative to each other for inducing a curved bend in the curvature controllable portion, characterised in that a means is provided for constraining the first and second members to move parallel to each other for inducing the curved bend in the curvature controllable portion.

70. A method as claimed in claim 69 in which the first and second members are disposed side by side and are slideably moveable axially relative to each other.

71. A method as claimed in claim 69 in which the means for constraining the first and second members to move parallel to each other is provided by a guide tongue extending laterally from the second member and being slideably engageable with an axially extending corresponding guide groove in the first member.
